(19) United States
(12) Patent Application Publication
(10) Pub. No.: US 2026/0263136 A1
(43) Pub. Date: Sep. 10, 2026

(54) CRYOABLATION SYSTEM AND METHOD FOR TREATING BREAST TUMOR EXCISION CAVITY MARGINS WITH ADAPTIVE CONTACT, TISSUE COMPRESSION, AND CONTROLLED CRYOGENIC TEMPERATURE DELIVERY

(71) Applicant: SenoGuard, Inc., Seal Beach, CA (US)

(72) Inventors: Greig Eric Altieri, Charlotte, NC (US); Diana Lee Tucker, Seal Beach, CA (US); Roberta Lee, Seal Beach, CA (US); Darius S. Francescatti, Barrington, IL (US); Sean Carroll, Pasadena, CA (US); Dan Wittenberger, Blainville (CA); John M. Baust, Seal Beach, CA (US)

(21) Appl. No.: 19/664,408

(22) Filed: Apr. 30, 2026

Related U.S. Application Data
(63) Continuation-in-part of application No. 18/548,064, filed on Feb. 28, 2022.
(60) Provisional application No. 63/154,561, filed on Feb. 26, 2021.
(60) Provisional application No. 63/797,648, filed on Apr. 30, 2025.
(60) Provisional application No. 63/797,656, filed on Apr. 30, 2025.

(51) Int. Cl. A61B 18/02; A61B 17/00; A61B 18/00; A61B 34/10; A61B 90/00

(52) U.S. Cl. CPC A61B 18/02; A61B 34/10; A61B 90/04; A61B 90/06; A61B 2017/00199; A61B 2017/00544; A61B 2017/00566; A61B 2017/00862; A61B 2017/00867; A61B 2017/00893; A61B 2018/00101; A61B 2018/00178; A61B 2018/00184; A61B 2018/00214; A61B 2018/00333; A61B 2018/00577; A61B 2018/00714; A61B 2018/00738; A61B 2018/00791; A61B 2018/0262; A61B 2034/101; A61B 2090/0463; A61B 2090/065; A61B 2218/007

(57) ABSTRACT

Cryoablation systems and methods for treating tissue surrounding a cavity created by breast tumor excision are disclosed. The invention provides both fixed-probe and expandable-probe configurations. The expandable probe incorporates one or more adaptive contact mechanisms that actively conform the probe surface to the irregular geometry of the surgical cavity. A tissue compression mechanism applies controlled compressive force to cavity wall tissue to eliminate interfacial air gaps, increase tissue density, and improve thermal conductivity at the probe-tissue interface. Associated methods include tumor removal, adaptive probe deployment, tissue compression, real-time lethal isotherm monitoring, and optional intraprocedural delivery of tissue fillers, cryo-enhancing agents, and/or patient-specific tumor antigens.

### CROSS-REFERENCE TO RELATED APPLICATIONS

This application is a continuation-in-part of U.S. Non-Provisional Patent Application No. Ser. No. 18/548,064, entitled “APPARATUS AND METHOD FOR MARGINAL ABLATION IN TISSUE CAVITY,” filed Aug. 25, 2023, which claims priority to PCT Application No. PCT/US 2022/018180, filed Feb. 28, 2022, and which claims benefit of U.S. Provisional Patent Application No. 63/154,561, filed Feb. 26, 2021, entitled “APPARATUS AND METHOD FOR ABLATION OF SOFT TISSUE SURROUNDING A BREAST CAVITY FOLLOWING LUMPECTOMY USING A FIXED PROBE,” all of which are incorporated herein by reference in their entireties for all purposes. This application also claims priority to U.S. Provisional Patent Application No. 63/797,648, filed Apr. 30, 2025, entitled “MINIMALLY INVASIVE TREATMENT OF SOFT TISSUE TUMORS” and claims priority to U.S. Provisional Patent Application No. 63/797,656, filed Apr. 30, 2025, entitled “CRYOABLATION SYSTEM AND METHOD FOR TREATING TISSUE SURROUNDING A CAVITY CREATED BY BREAST TUMOR EXCISION” the entirety of which is incorporated herein by reference.

### FIELD OF THE INVENTION

This invention relates generally to medical devices and methods. More particularly, this invention relates to methods and devices for treating a margin of soft tissue within the walls of a surgical cavity in a breast following the removal of a cancerous tumor.

### BACKGROUND OF THE INVENTION

With the advancement and increasing use of screening technologies, soft tissue cancers are being detected at earlier stages and smaller sizes. In particular, breast cancer is being detected earlier with screening mammography. The current standard of care for early-stage breast cancer typically involves surgical excision followed by radiation therapy. This approach, while effective, requires radiation treatment that can be invasive, painful, time-consuming, and potentially disfiguring.

The current standard of care for breast conservation is irradiation of the whole breast or a segment of the breast to eradicate any residual cancer cells following a lumpectomy or excision of a cancer. Since approximately 95% of recurrences occur at the site of the original tumor, post-operative radiation of the breast is the recommended local treatment in most breast cancers. However, radiation therapy has serious shortcomings, including gross overtreatment of tissue and potential significant complications. Short-term complications include skin burns, woody breast texture, tissue distortion, and shrinkage. Long-term complications include lymphedema of the arm, delayed cardiotoxicity, and occasionally, the development of a secondary non-breast cancer.

Various minimally invasive approaches have been developed to treat tumors, including cryoablation. Cryoablation involves the use of extreme cold to destroy targeted tissue and has been applied to various types of cancer including liver, lung, prostate, and breast tumors. Conventional cryoablation systems typically use rigid, non-deformable needle-like probes inserted percutaneously under image guidance. These systems deliver a cryogen to the probe tip to create an “ice ball” that freezes and destroys the surrounding tissue and can treat the solid tumor itself and not the tissue that once surrounded the tumor prior to excision.

Known devices for treating post-resection wound cavities, such as those described in the prior art, employ rigid, non-deformable applicator bodies that are sized and shaped to be positioned within the wound cavity. Such non-deformable bodies are incapable of conforming to the highly irregular, non-spherical geometries of surgically created breast cavities. Tumor excision cavities are rarely symmetric; they may present irregular lobes, recesses, and surface irregularities depending on the margin dissection technique, the anatomy of the patient, and the location and shape of the original tumor. When a non-deformable applicator body is inserted into such an irregular cavity, the mismatch between the rigid probe geometry and the cavity geometry inevitably results in regions of insufficient contact, intervening air or fluid gaps that thermally insulate the tissue from the device, and consequently, non-uniform thermal delivery and potential for incomplete margin treatment.

Furthermore, prior art devices for cavity treatment do not teach or suggest the delivery of cryogenic temperatures within a specific lethal temperature range calibrated for the destruction of breast cancer cells in cavity margin tissue. The destruction of cancer cells by freezing requires that the target tissue be cooled to a temperature sufficiently below the lethal isotherm threshold—generally accepted in the oncological cryosurgery literature as approximately −40° C. or lower at the target tissue boundary—and maintained at that temperature for a sufficient time. Superficial applicators that merely deliver “cold” temperatures, or that achieve cooling in the general range of 0° C. to −20°C., may not reliably achieve the cell-lethal temperatures required for complete margin sterilization. Prior art devices for wound cavity treatment do not disclose specific tissue-lethal cryogenic temperature ranges, temperature monitoring protocols for confirming achievement of lethal isotherms at the cavity margin, or systems for controlling cryogenic fluid delivery to ensure that a defined lethal temperature is reached at a specified depth from the cavity wall.

Known prior art cryocatheters used in cardiac ablation, such as endovascular devices, are designed for treating small, discrete tissue loci within blood vessels or cardiac chambers and differ fundamentally in structure and function from devices intended for treating the broad, irregular surface area of a post-excision breast tumor cavity. Cardiac cryocatheters are dimensioned to fit within endovascular lumens, employ contact-orientation sensing based on impedance or differential thermocouples to detect which side of a symmetric cylindrical catheter tip touches a vessel wall, and are not configured for or capable of providing uniform thermal treatment to an expansive, geometrically irregular, surgically created tissue cavity. The contact-orientation detection in cardiac cryocatheters is used merely to determine which side of the catheter tip is touching the vessel wall, not to monitor or confirm achievement of a cell-lethal isotherm throughout the entire surface area of an irregular breast tumor excision cavity.

Additionally, prior art devices used for treating wound cavities do not teach the use of active tissue compression to improve probe-to-tissue contact and to increase the density and thickness of the tissue at the cavity margin for more effective thermal conduction. Air gaps, seroma fluid pockets, and the natural tendency of tissue to retract away from the cavity surface during freezing are recognized problems that reduce the uniformity and depth of cryoablation. Active tissue compression addresses these problems by mechanically urging the cavity wall tissue into intimate contact with the probe surface and by compressing the tissue to reduce intercellular fluid volume, increase tissue density, and improve the thermal conductivity of the tissue-probe interface.

There remains a need for improved minimally invasive approaches that can effectively treat soft tissue tumors, particularly breast cancer, by delivering cryogenic temperatures within a defined, cell-lethal temperature range to an irregular post-excision cavity, while providing complete and uniform probe-to-tissue contact across the entire cavity surface, intraoperative monitoring of lethal isotherm achievement, active tissue compression, and enabling adjuvant therapies to be delivered in the same procedural setting.

### SUMMARY OF THE INVENTION

The present invention provides cryoablation systems and methods for treating the tissue surrounding a cavity created by the excision of a breast tumor. The systems and methods target and destroy any remaining cancer cells in the margin region of the surgical cavity by delivering cryogenic temperatures within a defined lethal temperature range to a specified depth of cavity margin tissue, providing a focused alternative to traditional whole-breast irradiation, while supporting intraprocedural delivery of pharmacologic agents, tissue surrogates, and patient-specific tumor antigens.

In one aspect, the invention provides a minimally invasive cryoablation device with an expandable probe that overcomes the conformity limitations of prior non-deformable applicator bodies. The expandable probe transitions from a contracted insertion state to an expanded treatment state and incorporates one or more adaptive contact mechanisms configured to conform the probe surface to the irregular geometry of the cavity walls, including differential expansion elements, tissue-sensing expansion mechanisms with integrated pressure sensors, multi-sectional independently expandable chambers, micro-textured surface patterns, shape-adaptive mesh frameworks of shape-memory alloy, vacuum-assisted contact enhancement with distributed micro-vacuum ports, articulating segments, dynamically adjustable expansion control, and individually targeted expansion point deployment based on pre-procedure imaging.

In another aspect, the invention provides a tissue compression mechanism integrated with the cryoablation probe or provided as a complementary system component, configured to mechanically compress the tissue surrounding the surgical cavity toward and against the probe surface, thereby eliminating interfacial air gaps and fluid pockets, increasing tissue density, and improving thermal conduction between the probe surface and the cavity margin tissue for more effective and uniform cryoablation.

In another aspect, the invention provides a fixed probe cryoablation system for supplying cryogenic energy within a defined lethal temperature range to a surgically created cavity. The fixed probe includes an exterior heat transfer surface shaped to maximize contact with the inner walls of the surgical cavity, at least one temperature sensing element mounted thereon or at a controlled distance therefrom, and a cryogenic fluid supply conduit and a cryogenic fluid return conduit. The probe is constructed of a rigid, biocompatible material such as stainless steel, cobalt-chrome alloy, titanium, or nickel-titanium alloy.

In yet another aspect, the invention provides a complete cryoablation system comprising either probe type coupled to a console that controls cryogenic fluid delivery, executes freeze-thaw-freeze cycles, monitors treatment progression through real-time temperature feedback, and confirms achievement of a lethal isotherm of −40° C. or lower, and more preferably −40° C. to −80° C., at a target depth of 0.5 to 2.0 cm from the inner surface of the cavity wall.

The invention further provides methods and treatments that can be delivered intraprocedurally, including: (a) injecting a non-toxic filler replicating the turgor of normal breast tissue; (b) injecting a non-toxic cryo-enhancing agent; (c) injecting a non-toxic filler accepting a tumor antigen preparation; (d) injecting a patient-specific antigen preparation; (e) removing injected agents after treatment; and (f) determining filler volume based on the labeled volume of the probe.

The invention provides significant clinical advantages including enabling treatment of small, low-grade tumors, reducing patient discomfort, disfigurement, and recovery time, and providing a procedure performable in an outpatient setting without general anesthesia.

### BRIEF DESCRIPTION OF THE DRAWINGS

The foregoing and other objects, features, and advantages of the invention will be apparent from the following more particular description of preferred embodiments as illustrated in the accompanying drawings, in which reference numerals refer to the same parts throughout the various views. The drawings are not necessarily to scale.

FIGS. 1A-1E illustrate in cross-section a breast and the sequential steps for treatment of breast cancer using a cryogenic probe system, including: (1A) cancer in the breast; (1B) surgical cavity after tumor excision; (1C) probe positioned in the cavity; (1D) cryoablation of the cavity margin; and (1E) the breast following removal of the probe and closure of the surgical tract;

FIG. 2 is a cross-sectional view of a fixed probe cryoablation device showing the tissue-contacting fixed probe supported by at least one co-axial tube on its exterior or embedded into its surface, and illustrating cryogenic fluid supply and return paths;

FIG. 3 is a cross-sectional view of a fixed probe cryoablation device with a unified cryogenic fluid supply or return path illustrating the coaxial inflow and outflow configuration;

FIG. 4 is a cross-sectional view of a fixed probe cryoablation device with a unified cryogenic fluid supply or return path through the center of the tissue-contacting fixed probe;

FIG. 5 is a cross-sectional view of a fixed probe cryoablation device further comprising a co-axial suction channel to remove excess air or fluid between the tissue-contacting fixed probe and the tissue cavity;

FIG. 6 illustrates two orthogonal views of an alternate fixed probe cryoablation device with a unified cryogenic fluid supply or return path through the length of the tissue-contacting fixed probe.

FIG. 7 illustrates two orthogonal views of the fixed probe cryoablation device of FIG. 6 with a temperature sensing probe for monitoring isotherm progression from the probe surface through the surrounding tissue, with representative temperature isotherms indicating achievement of the lethal temperature range at a target depth;

FIG. 8 is a perspective view of a minimally invasive cryoablation device with an expandable probe, showing a handle with controls, an outer tubular member, and an expandable probe element at the distal end;

FIG. 9 is a cross-sectional view of the distal end of the cryoablation device of FIG. 8, showing the expandable probe in a contracted state suitable for insertion;

FIG. 10 is a cross-sectional view of the distal end of the cryoablation device of FIG. 8, showing the expandable probe in a fully expanded state with expandable elements extending radially outward to contact cavity walls;

FIG. 11 is a schematic system diagram showing the cryoablation device connected to a console comprising a cryogen source, control unit, and display;

FIG. 12 illustrates a method of using the cryoablation device, depicting the expandable probe inserted into a cavity within breast tissue, in the expanded state, with the zone of ablation extending from the cavity walls into the surrounding tissue;

FIG. 13 is a schematic cross-sectional view illustrating the differential expansion element embodiment showing probe segments with different expansion profiles conforming to an irregular cavity geometry;

FIG. 14 is a schematic diagram illustrating the multi-sectional expansion zone embodiment showing independently addressable chambers and associated control lines from the handle;

FIG. 15 is an enlarged partial cross-sectional view of the expandable probe surface showing micro-textured surface features;

FIG. 16 is a perspective view of the shape-adaptive mesh framework embodiment in contracted state (left) and conforming to an irregular cavity (right);

FIG. 17 is a cross-sectional view of the vacuum-assisted contact enhancement embodiment showing micro-vacuum ports distributed across the probe surface and the associated vacuum lumen;

FIG. 18 is a perspective view of the articulating segment probe embodiment showing the individual segment joints and actuation cables;

FIG. 19 is a functional block diagram of the dynamically adjustable expansion control system illustrating signal flow between pressure sensors, the control unit, and the individual expansion actuators;

FIG. 20 is a schematic diagram illustrating the targeted expansion point deployment embodiment showing selective deployment of expansion elements based on pre-procedure imaging data overlay;

FIG. 21 is a schematic cross-sectional view illustrating the tissue compression mechanism embodiment, showing external compression elements urging cavity wall tissue against the probe surface, and depicting the resulting increase in tissue density and contact area; and

FIG. 22 is a graph of representative tissue temperature profiles at distances of 0, 0.5, 1.0, and 2.0 cm from the probe surface during a freeze-thaw-freeze cycle, illustrating achievement of the lethal isotherm of −40° C. at the target ablation depth.

### DETAILED DESCRIPTION OF THE INVENTION

Reference will now be made in detail to the preferred embodiments of the invention, examples of which are illustrated in the accompanying drawings. Like reference numerals refer to like elements throughout.

Fixed probe systems to deliver cryogenic cooling to the walls of a surgical cavity are described. In one embodiment, a fixed probe delivers cryogenic energy to lethally freeze a margin of soft tissue surrounding a surgical cavity in the breast to ablate or necrotize any remaining cancerous or precancerous cells. An additional configuration optionally includes a secondary suction channel to improve tissue contact, increase system efficiency, and reduce procedure time. Suitable cryogenic fluid agents include, without limitation, nitrous oxide, liquid nitrogen, supercritical nitrogen, helium, oxygen, and argon.

The fixed probe 22 may be constructed as a solid, hollow, or mesh member of spherical, ellipsoidal, ovoid, pyriform, or other geometry selected to approximate the shape of the surgical cavity. The interior of a hollow fixed probe member may be maintained at atmospheric pressure or under vacuum to insulate the probe interior and maximize thermal energy transfer to the surrounding tissue. The overall shape can be constructed as a smooth continuous surface or by tightly winding and coiling a small-diameter tube to approximate the desired shape.

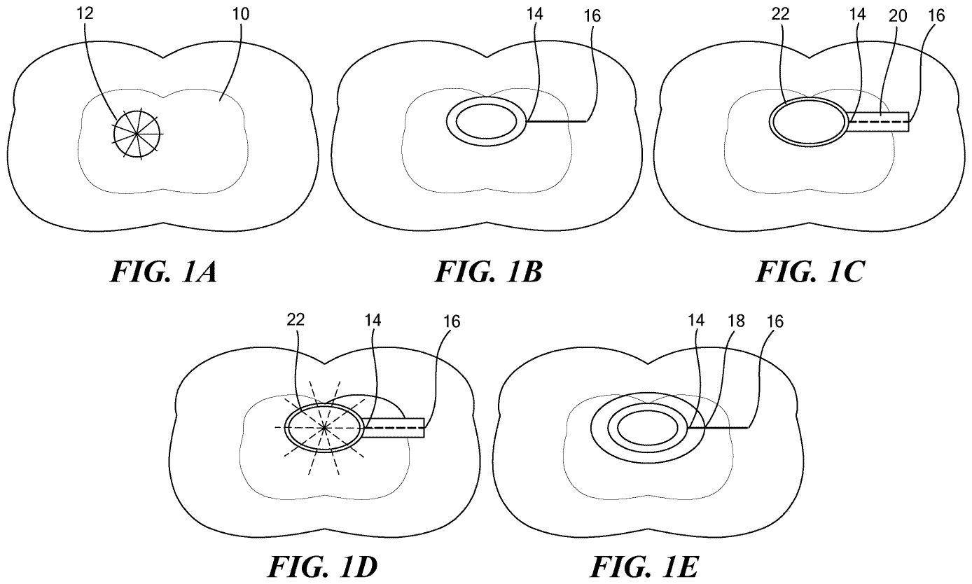

FIGS. 1A-1E illustrate sequential steps of treatment for surgical excision of a breast cancer followed by fixed probe cryoablation. FIG. 1A illustrates cancer 10 in breast 12. FIG. 1B illustrates surgical cavity 14 in breast 12 with surgical tract 16 after the cancer has been excised. FIG. 1C illustrates fixed probe system 20 placed within the surgical tract with fixed probe 22 in surgical cavity 14. FIG. 1D illustrates the fixed probe 22 with cryogenic fluid circulating and commencement of ablation. FIG. 1E illustrates the breast 12 after the fixed probe system has been removed and the surgical tract closed, showing that a margin of soft tissue 18 within the wall of the surgical cavity 14 has been ablated.

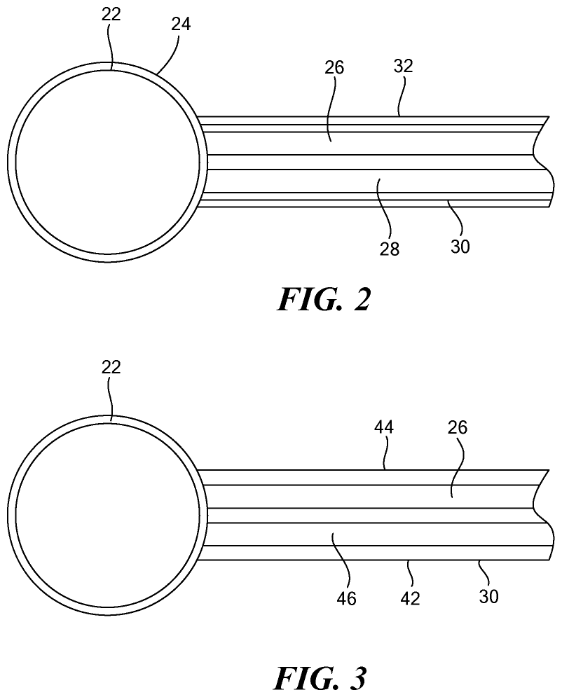

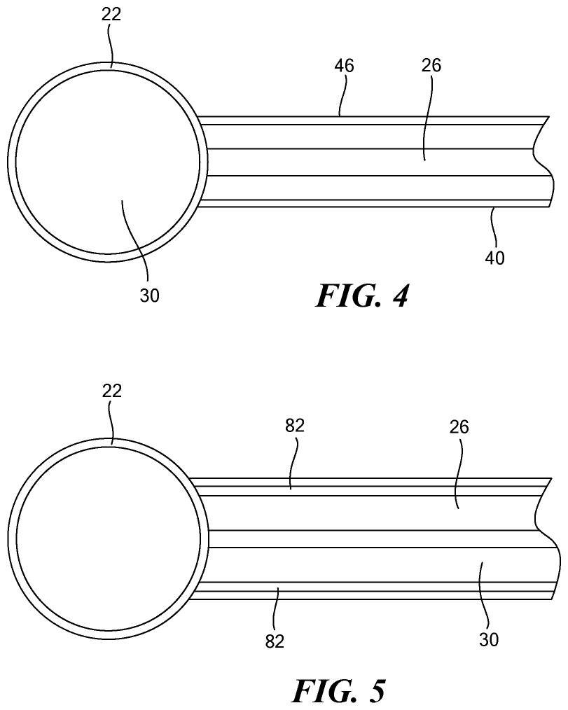

FIG. 2 illustrates a cross-sectional view of a fixed probe cryoablation device 20. The tissue-contacting fixed probe 22 is supported by at least one co-axial tube 24 on its exterior or embedded into its surface. Cryogenic fluid is infused into and circulated along the exterior of the tissue-contacting fixed probe 22, entering through cryogenic fluid supply tube 26 and, optionally, a second supply tube 28, and exiting through cryogenic fluid return tube 30 and, optionally, a second return tube 32. FIG. 3 illustrates a fixed probe cryoablation device 40 with a unified cryogenic fluid supply or return path. Cryogenic fluid enters through supply tube 26 and exits through return tube 30. Alternatively, flow direction may be reversed, entering through supply tube 42 and exiting through return tube 44 and optional secondary return tube 46. FIG. 4 illustrates a fixed probe cryoablation device 60 with the cryogenic fluid supply or return path through the center of the tissue-contacting fixed probe 22.

FIG. 5 illustrates a fixed probe cryoablation device 80 further incorporating a co-axial suction channel 82 around the periphery of the proximal portion of the device to remove excess air or fluid between the tissue-contacting fixed probe 22 and the tissue cavity. The suction channel 82 communicates with a vacuum source and is configured to draw tissue against the probe surface, eliminating thermally insulating air gaps and wound fluid pockets. Once intervening material has been removed and tissue contact has been established, the cryoablation process is initiated, ensuring intimate contact between the probe surface and the cavity wall for uniform energy transfer.

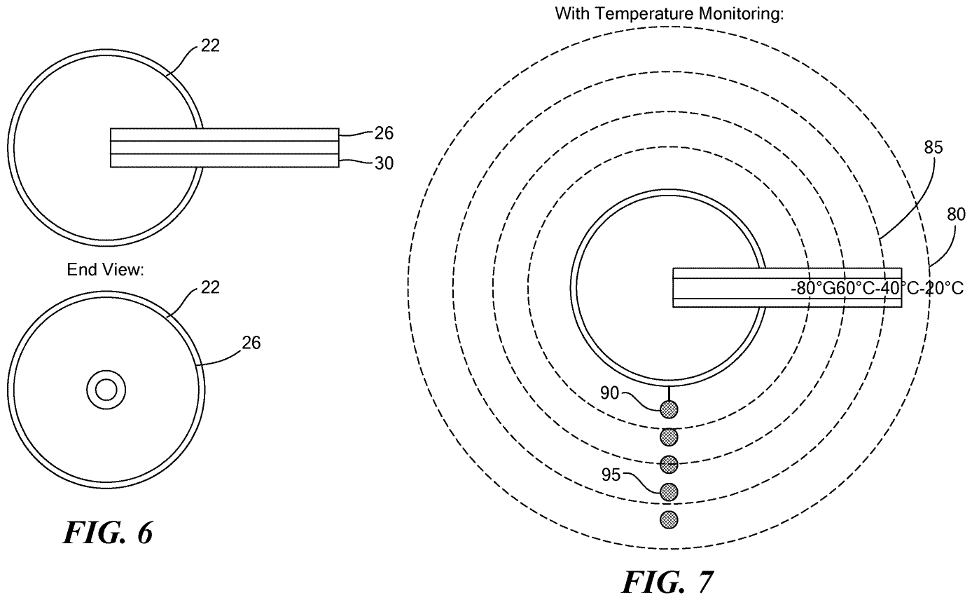

FIGS. 6 and 7 illustrate an alternate fixed probe cryoablation device 60 and the isotherm monitoring system. Temperature sensing probe 90 is inserted until it contacts the cryoablation device 22. Cryogenic fluid circulates through supply lumen 26 and exits through return lumen 30. The leading isotherm 80 will be warmer than all trailing isotherms, and the lethal isotherm 85 is selected based on the type of tumor being treated. The fixed probe system is coupled to a cryogenic fluid delivery system having one or more distal outflow ports and one or more inflow ports. The cooling system comprises a Joule-Thomson effect cooler or other system relying on expansion and phase change of a liquid passing through a valve, or alternatively an evaporative cooling system such as a single phase liquid cooling system.

The cryoablation systems and methods of the present invention are configured to deliver cryogenic temperatures within a specifically defined lethal temperature range that is calibrated to ensure reliable destruction of breast cancer cells and any residual precancerous cells in the cavity margin tissue. This temperature specification distinguishes the present invention from prior devices that deliver generic “cold” temperatures to body cavities without specifying or monitoring the achievement of cell-lethal conditions.

The console 50 and cryogenic fluid delivery system of the present invention are configured to cool the probe surface to a surface temperature in the range of −80° C. to −196° C., and more preferably −100° C. to −160° C., depending on the selected cryogenic agent. When argon gas is used as the cryogenic agent with a Joule-Thomson cooler, probe surface temperatures in the range of −120° C. to −140° C. are achievable. When liquid nitrogen is used, probe surface temperatures approaching −196° C. are achievable. When nitrous oxide is used as the cryogenic agent, probe surface temperatures in the range of −60° C. to −80° C. are achievable.

The clinically relevant parameter for margin ablation is the temperature achieved at the boundary of the target ablation zone within the cavity wall tissue, not the probe surface temperature. Breast cancer cells are reliably destroyed when the tissue temperature falls to −40° C. or below. Accordingly, the present invention defines a target lethal isotherm of −40° C. or lower, and preferably in the range of −40° C. to −80° C., that must be achieved at the desired ablation depth of 0.5 to 2.0 cm from the inner surface of the cavity wall. At a temperature of −40° C., ice crystal formation within intracellular and extracellular compartments causes irreversible mechanical and osmotic cell injury. At temperatures of −60° C. to −80° C., additional mechanisms including protein denaturation, lipid membrane disruption, and vascular stasis further ensure complete and permanent cell death. The present invention is specifically designed to achieve and confirm that the −40° C. or lower lethal isotherm has penetrated to the full target ablation depth of 0.5 to 2.0 cm before the freeze cycle is terminated.

To achieve the target lethal isotherm at the desired ablation depth, the probe surface must be maintained at its target surface temperature for a treatment duration determined by the system based on real-time temperature monitoring. For a target ablation depth of 1.0 cm with a probe surface temperature of −120° C., the typical treatment duration is in the range of 5 to 15 minutes per freeze cycle, depending on probe geometry, tissue vascularity, tissue composition, and the initial temperature of the tissue. For a target ablation depth of 2.0 cm, treatment durations of 10 to 25 minutes may be required. The console 50 calculates and displays the recommended treatment duration in real time based on the temperature sensor data and a computational model of isotherm propagation, and alerts the clinician when the target lethal isotherm has been achieved at the specified ablation depth.

The present invention further contemplates one or more freeze-thaw-freeze cycles to enhance cell destruction. During the first freeze cycle, ice crystal formation causes initial cell injury. During the thaw cycle, osmotic shifts and vascular reperfusion injury further damage cells. During the second freeze cycle, ice crystals re-form within a partially damaged cellular matrix, causing more extensive and complete cell death than the first freeze cycle alone. Each freeze-thaw-freeze cycle is executed under control of the console 50 and monitored by the temperature sensing system. The thaw cycle is defined as the period during which the probe surface temperature rises from its minimum to 0° C. or above, as measured by the probe surface temperature sensor. The second freeze cycle is initiated only after the control unit 54 confirms that the probe surface temperature has returned to 0° C. or above, to ensure that adequate thaw has occurred before refreezing.

The temperature sensing elements of the present invention, whether needle probes positioned at known depths within the tissue, surface probes on the probe element, or distributed sensors along a separate temperature sensing probe, provide real-time data that the console uses to confirm achievement of the lethal temperature range. The console 54 displays a graphical representation of the isotherm profile—showing tissue temperature as a function of depth from the probe surface—that is updated continuously during the freeze cycle. The display 56 indicates when the −40° C. isotherm has reached the target ablation depth, when the −60° C. and −80° C. isotherms have advanced, and when the minimum temperature at the deepest target tissue point has been achieved. This real-time isotherm visualization enables the clinician to make an informed, data-supported decision about when to terminate the freeze cycle, ensuring complete margin treatment without unnecessary prolongation of the procedure.

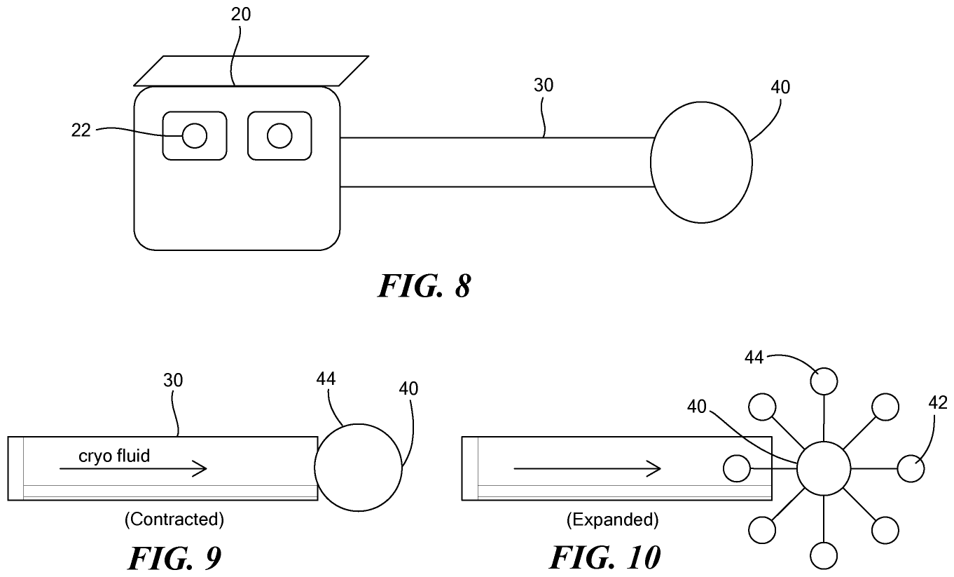

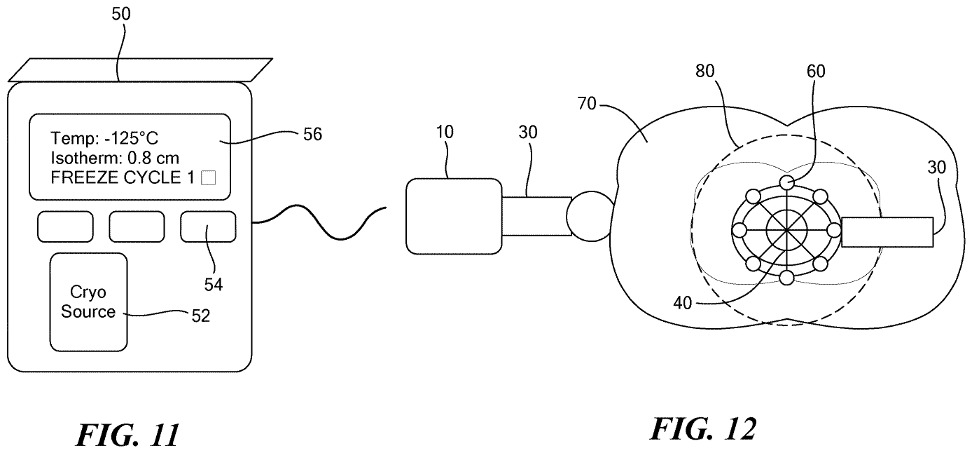

FIG. 8 shows a minimally invasive cryoablation device 10 comprising a handle 20, an outer tubular member 30 extending from the handle 20, and an expandable probe 40 disposed at the distal end of the outer tubular member 30. The handle 20 includes controls 22 for operating the device and is configured to be connected to a console (shown in FIG. 11) supplying cryogenic fluid and controlling operation of the device. The outer tubular member 30 defines a lumen through which cryogenic fluid may be delivered to the expandable probe 40. As shown in FIG. 9, the expandable probe 40 is shown in a contracted state suitable for insertion into a previously created cavity in tissue. FIG. 10 illustrates the expandable probe 40 in an expanded state, with expandable elements 42 extending radially outward to contact cavity walls. The expandable probe 40 includes a distal tip 44 that may be rounded to minimize tissue trauma during insertion and may also include sensors for monitoring temperature or other parameters during the procedure.

The expandable probe 40 of the present invention is structurally and functionally distinguished from prior art non-deformable applicator bodies in that it actively conforms its surface geometry to the irregular shape of the surgical cavity, rather than relying on the tissue to conform to the rigid probe geometry. This conformity is achieved through the adaptive contact mechanisms described in Sections D through M below and enables uniform probe-to-tissue contact across the entire cavity surface regardless of cavity geometry. The present invention is further distinguished from endovascular cardiac cryocatheters, which are dimensioned for insertion through blood vessels, employ contact-orientation sensing to detect which side of a symmetric cylindrical tip touches a vessel wall, and are not designed for, and cannot achieve, uniform thermal treatment of the broad, geometrically irregular surface of a post-excision breast tumor cavity.

FIG. 11 shows a system incorporating the cryoablation device 10 connected to a console 50 comprising a cryogen source 52, a control unit 54, and a display 56. The cryogen source 52 may contain argon gas, liquid nitrogen, or another suitable cryogenic fluid selected to achieve the target probe surface temperature range of −80° C. to −196° C. described in Section B. The control unit 54 regulates cryogenic fluid flow, executes freeze-thaw-freeze cycles, monitors temperature sensor data, and controls the adaptive expansion mechanisms described below. FIG. 12 illustrates the expandable probe 40 inserted into cavity 60 within breast tissue 70, creating a zone of ablation 80 extending from the cavity walls into surrounding tissue.

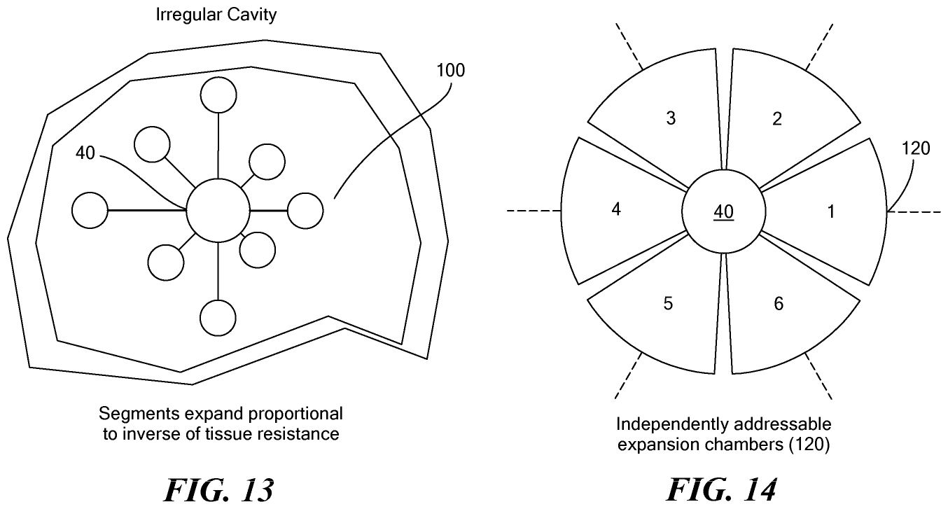

In one embodiment, illustrated schematically in FIG. 13, the expandable probe 40 incorporates differential expansion elements 100 comprising a plurality of discrete expansion segments, each independently capable of expanding to a different radial extent. Each differential expansion segment is fabricated from a material or structure—such as a variable-stiffness nitinol strut, a variable-wall-thickness elastomeric section, or a multi-layer composite member—that exhibits an expansion response proportional to the mechanical resistance encountered from the surrounding tissue. When a differential expansion segment meets a region of higher tissue resistance, the expansion of that segment is attenuated relative to segments encountering less resistance, allowing the probe as a whole to conform to the local surface geometry of the cavity. Differential stiffness may be achieved by varying the cross-sectional area, wall thickness, material composition, heat treatment profile, or mechanical geometry of individual segments.

In another embodiment, the expandable probe 40 incorporates a tissue-sensing expansion mechanism comprising a plurality of pressure sensors 110 integrated into the exterior surface of the expandable probe, each in signal communication with control unit 54. As the probe expands, the control unit continuously monitors the output of each pressure sensor 110 and compares the measured contact pressure against a target contact pressure range sufficient to ensure intimate thermal contact while remaining below a threshold that would cause mechanical injury to healthy tissue. When the measured contact pressure at a particular sensor location falls below the minimum of the target range, the control unit commands local expansion at that location. When it exceeds the maximum, the control unit limits or reverses local expansion at that location. The pressure sensors 110 may include piezoresistive pressure transducers, capacitive pressure sensors, fiber optic pressure sensors, or any other miniaturized pressure sensing technology suitable for operation at cryogenic temperatures.

In another embodiment, illustrated in FIG. 14, the expandable probe 40 is divided into a plurality of independently expandable sections or chambers 120, each in independent fluid communication with the cryogen delivery system or a separate pneumatic or hydraulic expansion fluid supply. Each section 120 can be selectively inflated or deflated via dedicated control lines extending through the outer tubular member 30 to the handle 20. The control unit 54 may selectively activate individual sections 120 based on pre-procedure imaging data representing the three-dimensional geometry of the surgical cavity, deploying expansion in zones where cavity wall contact is required while limiting expansion where the probe would otherwise press against skin or anatomical structures requiring protection.

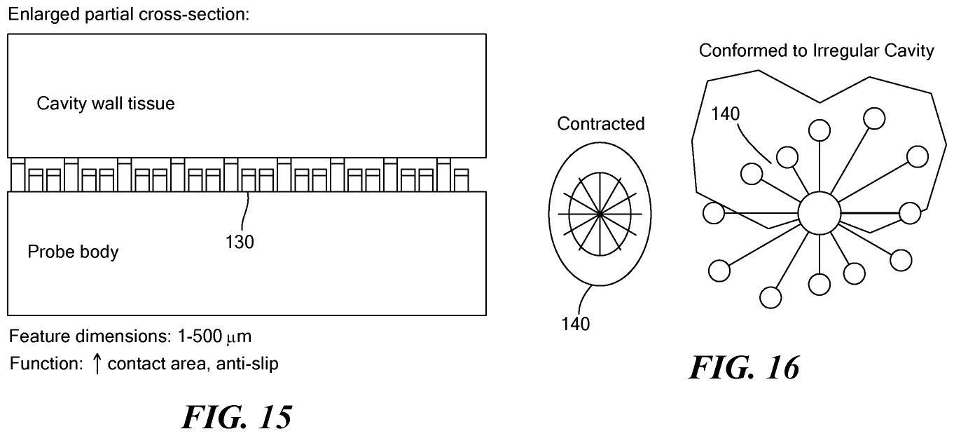

In another embodiment, illustrated in FIG. 15, the exterior surface of the expandable probe 40 incorporates a micro-textured surface pattern 130 comprising a plurality of microscopic features having dimensions on the order of 1 micron to 500 microns. The micro-textured surface pattern 130 serves two distinct functional purposes. First, by increasing the total contact surface area, the micro-textured surface pattern 130 increases the rate of heat transfer from the tissue to the probe surface, reducing treatment time for a given ablation depth. Second, the micro-textured surface pattern 130 creates mechanical interlocking between the probe surface and the tissue surface, resisting relative motion between the probe and the cavity wall during the freeze cycle, when tissue volumetric expansion due to ice formation may otherwise displace the probe from its desired position. The micro-textured surface pattern 130 may be applied by laser ablation, chemical etching, electro-discharge machining, additive manufacturing, coating deposition, or mechanical embossing.

In another embodiment, illustrated in FIG. 16, the expandable probe 40 comprises a shape-adaptive mesh framework 140 formed from a network of shape-memory alloy struts or wires, such as nitinol, arranged in a three-dimensional mesh geometry. In the contracted state, the mesh framework 140 is collapsed for insertion through the tissue tract. Upon deployment within the cavity, the mesh framework 140 transitions to its thermally preset expanded shape. Unlike a non-deformable body, the shape-adaptive mesh framework 140 deforms locally to accommodate protrusions, recesses, and surface irregularities of the cavity wall. The open mesh geometry permits direct fluid contact between the cryogenic fluid circulating within the mesh and the tissue at the interstices, enhancing cryogenic energy delivery. The mesh may be coated with a thermally conductive biocompatible material and may incorporate the micro-textured surface patterns described above.

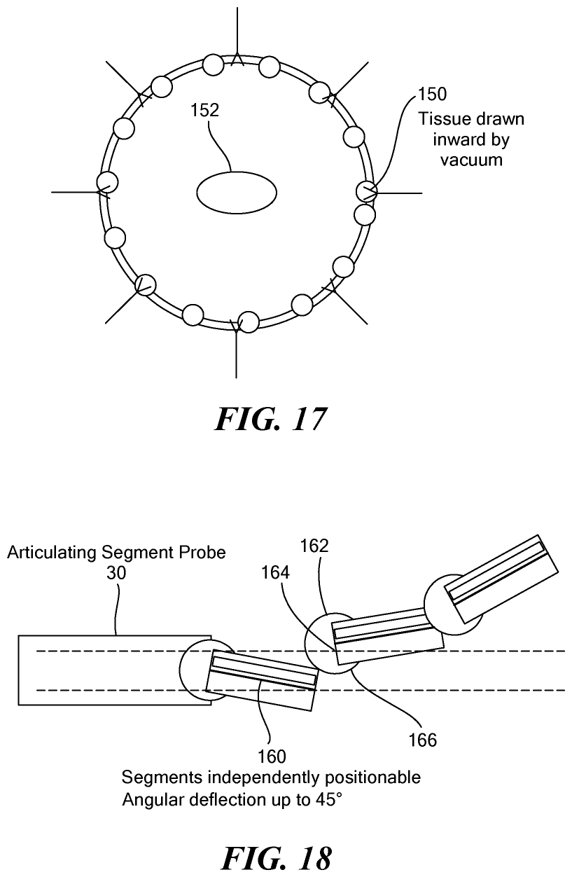

In another embodiment, illustrated in FIG. 17, the expandable probe 40 incorporates a vacuum-assisted contact enhancement system comprising a plurality of micro-vacuum ports 150 distributed across the exterior surface of the probe, each communicating with a vacuum lumen 152 extending through the outer tubular member 30 to a vacuum source. When negative pressure is applied through the vacuum lumen 152, the surrounding tissue is actively drawn against the probe surface, eliminating air gaps and fluid pockets. The micro-vacuum ports 150 may be selectively activated or deactivated by the control unit 54 based on pressure sensor feedback, applying vacuum selectively to regions where contact is not achieved by expansion alone.

In another embodiment, illustrated in FIG. 18, the expandable probe 40 comprises a plurality of articulating segments 160 connected by pivot joints 162, the segments being independently positionable by actuation cables 164 extending through the outer tubular member 30 to actuation controls at the handle 20. Each segment 160 may incorporate its own cryogenic fluid delivery channel, temperature sensor, pressure sensor, and micro-textured surface features. Adjacent segments 160 are separated by compliant interconnects 166 that permit angular deflection between segments of up to 45 degrees relative to the central axis of the probe. The articulating segment design is particularly suited for elongated or irregular cavities where a single expanding element cannot simultaneously contact all cavity walls.

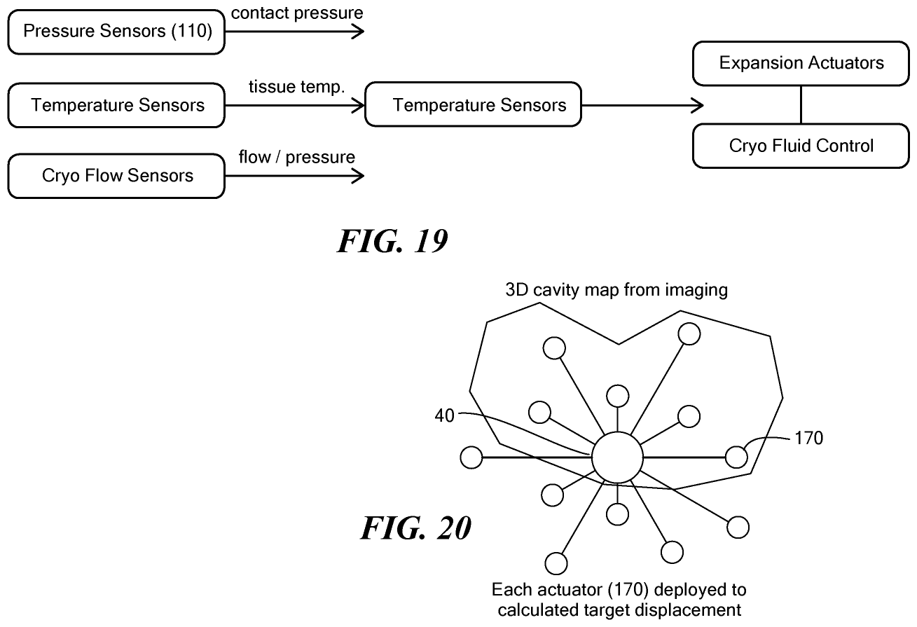

In another embodiment, illustrated in FIG. 19, the expandable probe 40 incorporates a dynamically adjustable expansion system in which the control unit 54 continuously monitors contact pressure sensor data from sensors 110, tissue temperature data from temperature sensors at the probe surface and at distances within the tissue, and cryogenic fluid flow rate and pressure data from the cryogen delivery system. Based on this multi-parameter monitoring, the control unit 54 continuously adjusts the expansion state during the treatment procedure. As tissue freezes, it becomes mechanically stiffer and may contract slightly, potentially reducing contact pressure. The dynamically adjustable expansion system detects such changes in real time and responds by increasing expansion in affected zones to maintain adequate contact pressure throughout the freeze cycle, thereby ensuring continuous and uniform thermal contact as the tissue transitions from its unfrozen to frozen state.

In another embodiment, illustrated in FIG. 20, the expandable probe 40 incorporates a plurality of individually deployable expansion points or actuators 170, each independently displaceable to a calculated target displacement position derived from a three-dimensional map of the surgical cavity obtained from pre-procedure imaging data (ultrasound, mammography, MRI, or CT). The control system uses this three-dimensional cavity map to calculate, for each expansion point 170, the displacement required to bring that expansion point into contact with the corresponding point on the cavity wall. The control unit 54 then deploys each expansion point 170 to its calculated target displacement position, achieving a customized three-dimensional expansion profile that conforms to the actual pre-measured geometry of the surgical cavity.

In another embodiment, the exterior surface layer of the expandable probe 40 is formed from one or more compliant bio-mimetic materials 180 whose mechanical compliance properties are matched to those of the surrounding breast tissue. Suitable compliant bio-mimetic materials include, without limitation: hydrogel composites with controlled cross-link density; fiber-reinforced elastomers in which fiber orientation and density replicate the anisotropic mechanical behavior of breast tissue; auxetic materials having a negative Poisson ratio; interpenetrating polymer network composites; and porous metallic foams with controlled porosity and pore geometry. By matching the mechanical compliance of the probe surface to that of the surrounding tissue, the compliant bio-mimetic material layer 180 distributes contact pressure uniformly across the probe-tissue interface, reducing peak contact stresses and promoting uniform cryogenic heat transfer.

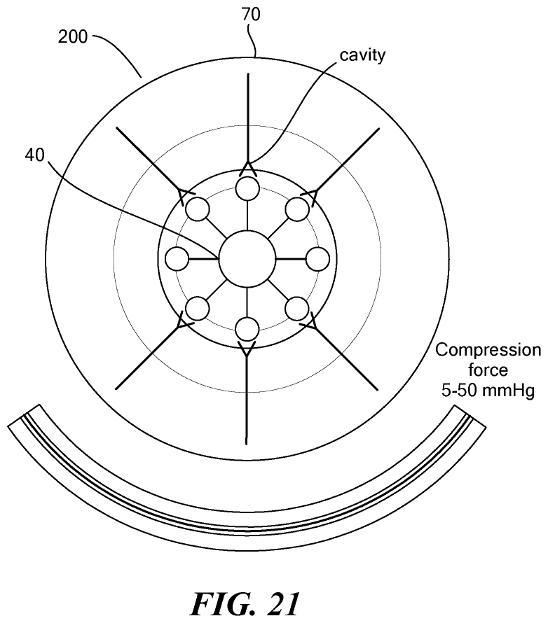

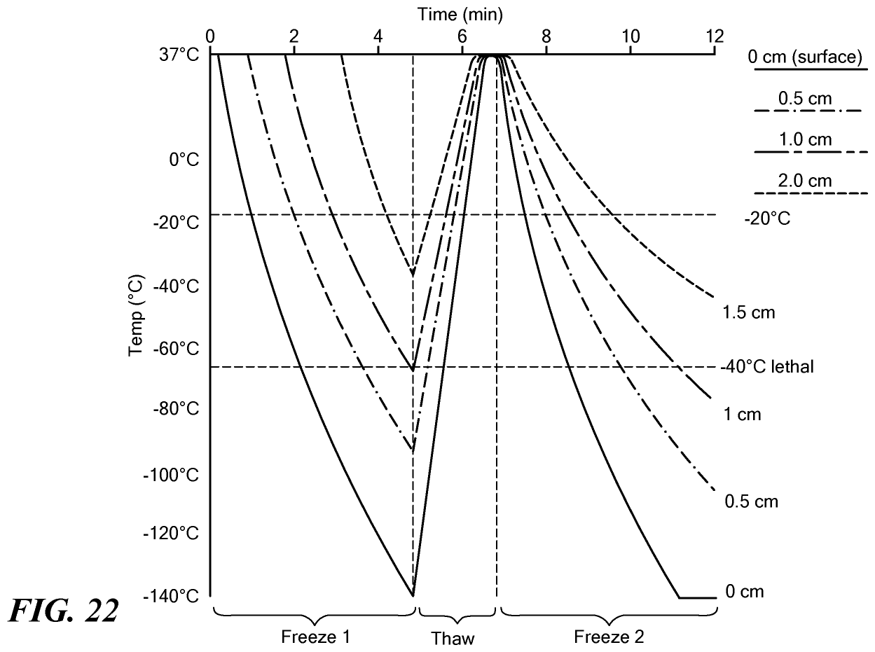

In another embodiment, illustrated in FIG. 21, the cryoablation system of the present invention incorporates a tissue compression mechanism configured to mechanically compress the tissue of the surgical cavity toward and against the exterior surface of the cryoablation probe. Tissue compression serves two distinct and synergistic clinical functions that are not achievable with prior art devices. First, compression eliminates the interfacial air gaps and wound fluid pockets—seroma, hematoma, and surgical irrigation fluid—that naturally accumulate within the surgically created cavity and that would otherwise thermally insulate portions of the cavity wall from the probe surface, preventing those portions from reaching the lethal temperature range. Second, compression increases the physical density of the tissue immediately adjacent to the probe surface by reducing intercellular and interstitial fluid volume, thereby increasing the volumetric density of cells, increasing the thermal conductivity of the tissue-probe interface, reducing the distance between the probe surface and target cells, and providing a greater mass of tissue at an elevated density within the lethal isotherm zone for more complete and uniform cell destruction.

The tissue compression mechanism may be implemented in one or more of the following configurations. In a first configuration, an external compression shell 200 or cup-shaped member is positioned around the exterior of the breast, enclosing at least the portion of the breast containing the surgical cavity, and a compressive force is applied to the exterior of the breast by the compression shell 200 to urge the cavity wall tissue radially inward toward the probe surface. The compressive force may be applied mechanically, pneumatically, or by adjustable straps or bladders. In a second configuration, the probe itself comprises an expandable compression element on its exterior surface, distinct from the cryogenic heat transfer surface, that can be selectively expanded to apply controlled compressive pressure to the cavity walls prior to and during the cryoablation procedure. In a third configuration, a vacuum-assisted tissue apposition system, as described in Section I above, is operated at a higher negative pressure sufficient to draw the cavity wall tissue into mechanical contact with the probe surface, providing both the contact-enhancement and tissue-compression functions simultaneously.

The degree of tissue compression applied by the tissue compression mechanism is controlled by the control unit 54 based on feedback from the pressure sensors 110 distributed across the probe surface. The target contact pressure for tissue compression is in the range of 5 to 50 mmHg, which is sufficient to eliminate interfacial air gaps and increase tissue density without causing ischemic injury to the compressed tissue. The tissue compression may be initiated prior to the commencement of cryoablation and maintained throughout the freeze cycle, and may be released during the thaw cycle to allow reperfusion of the compressed tissue. The compression-and-release cycle may be coordinated with the freeze-thaw-freeze cycle to maximize cell destruction by alternating between periods of compression-enhanced cryoablation and reperfusion-injury-inducing thaw.

Any two or more of the adaptive contact mechanisms described above may be combined in a single probe embodiment. In one particularly advantageous combination, the probe 40 incorporates: (i) a shape-adaptive mesh framework 140 as the primary structural expansion element; (ii) micro-textured surface features 130 applied to the mesh wire surfaces; (iii) micro-vacuum ports 150 distributed across the mesh to actively draw tissue into intimate contact; (iv) integrated pressure sensors 110 providing contact feedback to the control unit; (v) dynamically adjustable expansion control responding to pressure sensor data and tissue temperature changes during the freeze cycle; and (vi) an external tissue compression shell 200. This combined adaptive contact system achieves a level of cavity wall conformity and thermal contact uniformity that is unattainable by any single mechanism operating independently. As used herein, “substantially complete thermal contact” refers to probe-to-tissue apposition sufficient to eliminate thermally insulating air or wound fluid gaps over the treated cavity wall, which in certain embodiments is confirmed by pressure sensor feedback (e.g., contact pressures within a target range such as 5 to 50 mmHg at a majority of monitored locations) and/or by vacuum-assisted apposition indicating removal of intervening air or fluid.

The present invention provides a method to treat a patient after lumpectomy by administering intraprocedurally cryogenic temperatures within the lethal temperature range described in Section B to the created surgical cavity using a surgical probe and a cryogen delivery system. The method treats the margin of the surgically created tissue cavity by exposing it to cryogenic temperatures immediately after the surgical removal of a benign or malignant growth. The method may also involve treating at least a portion of the incision channel from the skin to the surgical cavity by exposing at least a portion of the refrigerant conduit; a slidable insulator may be positioned over at least a portion of the refrigerant conduit to protect the skin from unwanted cryogenic exposure.

For the minimally invasive expandable probe embodiment, the method generally includes: (a) optionally applying the tissue compression mechanism prior to probe insertion to consolidate the cavity geometry; (b) inserting the expandable cryoablation probe into the cavity; (c) deploying the adaptive contact mechanism to establish contact between the probe surface and the cavity wall; (d) applying tissue compression during cryoablation; and (h) activating cryoablation to deliver cryogenic temperature within the lethal temperature range of −40° C. to −80° C. at the target ablation depth of 0.5 to 2.0 cm.

The procedure may involve one or more freeze-thaw-freeze cycles controlled based on intraoperative temperature monitoring, confirming achievement of the −40° C. or lower lethal isotherm at the target ablation depth before terminating each freeze cycle. After completing the cryoablation, the probe is contracted and removed from the cavity. The method may include injecting a non-toxic filler replicating the turgor of normal breast tissue, visible under fluoroscopy, ultrasound, and/or infrared imaging. Examples of such fillers include sterile saline; biocompatible hydrogels (including polyethylene glycol-based and polysaccharide-based hydrogels); collagen or gelatin-based matrices; radiopaque or echogenic formulations, and their own fat cells from fatty areas of the body. In certain embodiments, a cryo-enhancing agent is injected, including hypertonic saline solutions or other agents that promote ice nucleation or enhance cryoinjury. In certain embodiments, a patient-specific tumor antigen preparation is administered intraprocedurally, including autologous tumor lysate or other antigenic material prepared using standard techniques and optionally formulated in a biocompatible carrier, and may further include injecting a patient-specific antigen or other pharmacologic agent effective against the cancer being treated.

The progression of cryoablation treatment is monitored by directly measuring the temperature in the tissue at a desired distance from the probe surface. A needle temperature probe may be affixed to the heat exchange element at a known distance from the surface, or movably attached so the operator can vary the penetration depth. Additionally, one or more surface temperature probes may be affixed to the probe surface or refrigerant conduit. The control unit computes the isotherm profile based on these measurements and the probe geometry, and determines when the lethal isotherm of −40° C. or lower has reached the target ablation depth of 0.5 to 2.0 cm. In the dynamically adjustable expansion embodiment, the temperature sensor data simultaneously drives both the isotherm monitoring function and the adaptive expansion control function, providing fully integrated thermal and mechanical management of the treatment procedure.

While the invention is particularly well-suited for treating breast cancer following lumpectomy, it may also be applied to other soft tissue tumors where a minimally invasive approach is desirable, including small tumors in the liver, kidney, lung, or other soft tissue locations. Any elements described herein as singular can be pluralized. The above-described configurations, elements, assemblies, and methods can be combined and modified in any combination. Reference is made to U.S. 2021/0153920, which has common inventorship herewith, the full disclosure of which is incorporated herein by reference.

1. A minimally invasive cryoablation device for treating tissue surrounding a surgically created cavity following excision of a breast tumor, the device comprising:
a handle;
an outer tubular member extending from the handle and defining a cryogenic fluid lumen, the outer tubular member having a proximal end and a distal end;
an expandable probe disposed at the distal end of the outer tubular member, the expandable probe being transitionable between a contracted state in which the expandable probe is sized for insertion through a tissue tract into the surgically created cavity, and an expanded state; and
a cryogen delivery system in fluid communication with the expandable probe via the cryogenic fluid lumen, the cryogen delivery system configured to deliver a cryogenic fluid to the expandable probe to cool tissue surrounding the cavity to a lethal tissue temperature of −40° C. or lower at a target ablation depth of 0.5 to 2.0 cm from an inner wall of the cavity;
wherein the expandable probe comprises at least one adaptive contact mechanism configured to conform the probe surface to an irregular geometry of the cavity walls to ensure substantially complete thermal contact between the probe surface and the cavity wall tissue across the entire inner surface of the cavity, as confirmed by at least one of: (i) contact pressure data from a plurality of pressure sensors distributed across the probe surface indicating contact pressure within a target range at a majority of sensor locations during a freeze cycle; and (ii) vacuum-assisted apposition eliminating intervening air or wound fluid gaps between the probe surface and the cavity wall.

2. The device of claim 1, wherein the at least one adaptive contact mechanism comprises differential expansion elements comprising a plurality of discrete expansion segments each independently expandable to a radial extent that is inversely proportional to the mechanical resistance encountered from cavity wall tissue at the corresponding location, whereby the expandable probe passively conforms to the cavity wall geometry without imposing uniform radial displacement on all cavity wall regions.

3. The device of claim 1, wherein the at least one adaptive contact mechanism comprises a tissue-sensing expansion mechanism comprising:
a plurality of pressure sensors distributed across an exterior surface of the expandable probe, each pressure sensor configured to measure local contact pressure between the probe surface and adjacent tissue; and
a control unit in signal communication with the plurality of pressure sensors and configured to independently modulate local expansion of the expandable probe at each pressure sensor location based on measured contact pressure to maintain contact pressure within a target range of 5 to 50 mmHg throughout a freeze cycle.

4. The device of claim 1, wherein the at least one adaptive contact mechanism comprises a multi-sectional expansion zone structure comprising a plurality of independently expandable chambers, each chamber in independent fluid communication with a corresponding control line extending through the outer tubular member, the independently expandable chambers being selectively activatable based on a three-dimensional geometric map of the surgically created cavity derived from pre-procedure imaging.

5. The device of claim 1, wherein an exterior surface of the expandable probe comprises a micro-textured surface pattern having feature dimensions in the range of 1 micron to 500 microns, the micro-textured surface pattern configured to increase thermal contact surface area between the expandable probe and the cavity wall tissue and to mechanically resist relative displacement between the expandable probe and the cavity wall during ice formation in the freeze cycle.

6. The device of claim 1, wherein the at least one adaptive contact mechanism comprises a shape-adaptive mesh framework formed from a network of shape-memory alloy struts arranged in a three-dimensional open mesh geometry, the shape-adaptive mesh framework configured to deform locally to accommodate surface irregularities of the cavity wall and to permit direct contact between cryogenic fluid and tissue through interstices of the mesh.

7. The device of claim 1, wherein the at least one adaptive contact mechanism comprises a vacuum-assisted contact enhancement system comprising:
a plurality of micro-vacuum ports distributed across the exterior surface of the expandable probe; and
a vacuum lumen extending through the outer tubular member in fluid communication with each of the plurality of micro-vacuum ports and connectable to a vacuum source, wherein negative pressure applied through the vacuum lumen draws cavity wall tissue against the probe surface to eliminate thermally insulating air gaps and wound fluid pockets between the probe surface and the cavity wall.

8. The device of claim 1, further comprising a tissue compression mechanism configured to apply a controlled compressive force to tissue surrounding the surgically created cavity to mechanically urge the cavity wall tissue against the probe surface, the tissue compression mechanism being operative to:
(i) eliminate interfacial air gaps and wound fluid pockets between the probe surface and the cavity wall; and
(ii) increase tissue density at the cavity wall to improve thermal conductivity of the probe-tissue interface during cryoablation.

9. The device of claim 8, wherein the tissue compression mechanism comprises at least one of:
(i) an external compression shell positionable around the breast and configured to apply an inwardly directed compressive force to the exterior of the breast to urge cavity wall tissue toward the probe surface; and
(ii) an expandable compression element on an exterior surface of the probe configured to apply controlled compressive pressure directly to the cavity walls, the compressive pressure being in the range of 5 to 50 mmHg.

10. The device of claim 1, further comprising a control unit configured to:
continuously monitor contact pressure data from pressure sensors distributed across the expandable probe surface and tissue temperature data from at least one temperature sensor during a cryoablation procedure; and
dynamically adjust expansion of the expandable probe during the procedure in response to changes in tissue mechanical properties caused by phase transition of tissue water to ice, thereby maintaining substantially uniform contact pressure as the tissue transitions from its unfrozen to frozen state.

11. The device of claim 1, wherein the at least one adaptive contact mechanism comprises a plurality of individually deployable expansion actuators, each independently displaceable under control of a control unit to a target displacement position derived from a three-dimensional geometric map of the surgically created cavity obtained from pre-procedure imaging data, whereby the expandable probe achieves a patient-specific non-uniform expansion profile conforming to the pre-measured cavity geometry.

12. The device of claim 1, wherein an exterior surface layer of the expandable probe is formed from a compliant bio-mimetic material having a stress-strain response approximating that of breast tissue within a contact pressure range of 5 to 50 mmHg, the compliant bio-mimetic material selected from the group consisting of: hydrogel composites, fiber-reinforced elastomers, auxetic materials having a negative Poisson ratio, interpenetrating polymer network composites, porous metallic foams, and combinations thereof.

13. A surgical cryoablation device for treating a marginal tissue region in a surgically created tissue cavity following excision of a breast tumor, the device comprising:
a shaft;
a fixed-shape, rigid probe element mounted on a distal region of the shaft, the probe element having an exterior heat transfer surface shaped and sized to make contact with an inner surface of the surgically created tissue cavity when placed therein, the probe element comprising a biocompatible material selected from the group consisting of stainless steel, cobalt-chrome alloy, titanium, nickel-titanium alloy, and combinations thereof;
a cryogenic fluid supply conduit and a cryogenic fluid return conduit each coupled to the probe element, the cryogenic fluid supply conduit delivering a cryogenic fluid to circulate along the exterior heat transfer surface via the cryogenic fluid return conduit;
at least one temperature sensing element configured to measure tissue temperature at a defined depth from the exterior heat transfer surface to confirm achievement of a lethal tissue temperature of −40° C. or lower at the target ablation depth; and
a suction conduit positioned exterior to the probe element and in fluid communication with a vacuum source and configured to remove air or wound fluid between the exterior heat transfer surface and the tissue cavity to ensure intimate thermal contact between the probe element and the cavity wall.

14. The device of claim 13, further comprising a slidable insulation element positionable over at least a portion of the cryogenic fluid supply conduit to selectively expose a desired portion of the conduit to surrounding tissue, thereby enabling selective cryotreatment of at least a portion of a tissue tract extending from skin to the surgically created cavity while protecting skin from cryogenic exposure.

15. A cryoablation system for treating a tissue cavity created by excision of a breast tumor, the system comprising:
a cryoablation probe configured to make thermal contact with inner walls of the tissue cavity, the probe comprising at least one adaptive contact mechanism configured to conform the probe surface to an irregular geometry of the cavity walls;
a tissue compression mechanism configured to apply a compressive force to tissue surrounding the cavity to eliminate interfacial air gaps, increase tissue density, and improve thermal conductivity at the probe-tissue interface;
a console connected to the cryoablation probe by one or more fluid and electrical umbilicals, the console comprising a cryogenic fluid source, a control unit, and a display;
and
a temperature monitoring system comprising at least one temperature sensing element positioned to measure tissue temperature at a target ablation depth during cryoablation, the control unit being programmed to confirm achievement of a lethal tissue temperature of −40° C. or lower at the target ablation depth and to display a real-time graphical representation of isotherm progression from the probe surface through the surrounding tissue.

16. The system of claim 15, wherein the control unit is programmed to execute one or more freeze-thaw-freeze cycles, each freeze cycle being terminated only after the temperature monitoring system confirms that the lethal tissue temperature of −40° C. or lower has been achieved at the target ablation depth, and each thaw cycle being defined by a return of the probe surface temperature to 0° C. or above as measured by a probe surface temperature sensor, before initiation of a subsequent freeze cycle.

17. A method for treating tissue surrounding a surgically created cavity following excision of a soft tissue tumor in a breast, the method comprising:
creating the cavity by removing at least a portion of a tumor;
inserting a cryoablation probe comprising at least one adaptive contact mechanism into the cavity;
deploying the at least one adaptive contact mechanism to conform the probe surface to the geometry of the cavity walls and establish substantially complete thermal contact between the probe surface and the cavity wall tissue;
applying compressive force to tissue surrounding the cavity to eliminate interfacial air gaps and increase tissue density at the probe-tissue interface; and
activating cryoablation to cool tissue surrounding the cavity to a lethal tissue temperature of −40° C. or lower at a target ablation depth of 0.5 to 2.0 cm from the inner surface of the cavity wall.

18. The method of claim 17, wherein deploying the at least one adaptive contact mechanism comprises:
monitoring contact pressure between the probe surface and the cavity wall at a plurality of locations using pressure sensors distributed across the probe surface; and
independently adjusting local expansion of the probe at each location based on monitored contact pressure to maintain contact pressure within a target range of 5 to 50 mmHg throughout activation of cryoablation;
wherein activating cryoablation further comprises monitoring tissue temperature at the target ablation depth in real time and terminating each freeze cycle only after confirming that the lethal tissue temperature of −40° C. or lower has been achieved at the target ablation depth.

19. The method of claim 17, wherein activating cryoablation comprises executing at least two freeze-thaw-freeze cycles, each freeze cycle delivering cryogenic fluid to maintain a probe surface temperature in the range of −80° C. to −196° C. for a duration sufficient to advance the −40°C lethal isotherm to the target ablation depth, and each thaw cycle comprising cessation of cryogenic fluid delivery and confirmation that the probe surface temperature has returned to 0° C. or above before initiation of the next freeze cycle.

20. The method of claim 17, wherein deploying the at least one adaptive contact mechanism comprises:
obtaining a three-dimensional geometric map of the surgically created cavity from pre-procedure imaging;
independently deploying each of a plurality of individually addressable expansion actuators of the probe to a calculated target displacement position derived from the three-dimensional geometric map; and
confirming substantially complete cavity wall contact using pressure sensors distributed across the probe surface before activating cryoablation;
the method further comprising injecting, after completion of cryoablation, at least one of: a non-toxic tissue filler replicating the turgor of normal breast tissue, a cryo-enhancing agent, and a patient-specific tumor antigen preparation.
